(19) United States
(12) Patent Application Publication
(10) Pub. No.: US 2009/0012601 A1
(43) Pub. Date: Jan. 8, 2009

(54) STENT AND CATHETER ASSEMBLY AND METHOD FOR TREATING BIFURCATIONS

(75) Inventors: Cynthia K. Siu, San Francisco, CA (US); Thomas Ray Hatten, Los Altos, CA (US); Brenna K. Hearn, San Francisco, CA (US); Sushila W. Singh, Newark, CA (US); Han Le Juanta, Milpitas, CA (US); Santosh Prabhu, Sunnyvale, CA (US); Bjorn Svensson, Gilroy, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS, INC., Santa Clara, CA (US)

(21) Appl. No.: 11/773,766

(22) Filed: Jul. 5, 2007

(51) Int. Cl. A61F 2/82

(52) U.S. Cl. 623/1.35

(57) ABSTRACT

An improved stent system for treating a bifurcation. Up to three stents are mounted on two parallel balloon catheters wherein a proximal stent is disposed about both balloons while two distal stents are each disposed about one of the balloons. Such configuration enhances the flexibility of the assembly to facilitate its advancement through tortuous vasculature, while the structural independence of the stents maximizes the systems ability to spread into a wide range of bifurcation angles. A single link between each distal stent and the proximal stent may be relied upon to maintain relative stent position during deployment.

### BACKGROUND OF THE INVENTION

The invention relates to stents and stent delivery and deployment assemblies for use at a bifurcation and, more particularly, for repairing diseased blood vessels at bifurcations. The invention further relates to methods for the delivery and implantation of stents at bifurcations.

Stents are expandable hollow structures, generally cylindrical in shape, that are used to repair blood vessels that are diseased. In use, a stent is advanced through the vasculature to the diseased area of a vessel and expanded so as to provide an unobstructed pathway for blood flow therethrough. It can also be combined with pharmaceuticals to improve healing and slow restenosis.

Repair of vessels that are diseased at a bifurcation is particularly challenging since the stent must be precisely positioned, provide adequate coverage of the diseased area, maintain vessel patency in order to allow adequate blood flow to reach the myocardium and provide access to any additional diseased area that may be located distally to the bifurcation. A stent that provides coverage to the vessel at the diseased portion, yet extends across one of the lumens at the bifurcation, would serve to treat the diseased area but may compromise blood flow to areas downstream of the bifurcation. Additionally, unapposed or poorly apposed stent elements may further promote lumen compromise during neointimal formation and healing by causing restenosis and which may require further procedures. Moreover, by extending across one of the lumens at the bifurcation, the stent may block access to further interventional procedures.

Conventional stents are designed to repair areas of blood vessels that are spaced apart from bifurcations, and therefore, a variety of problems may arise when attempting to use them to treat lesions at a bifurcation. In treating bifurcations, conventional stents are normally deployed so that the entire stent is either in the parent vessel or the proximal portion of the stent is in the parent vessel and the distal portion is located in the side branch vessel. Either the side branch vessel (former case) or the parent vessel (later case), would thereby become “jailed” by the stent struts. This technique repairs one vessel at the bifurcation at the expense of jailing or obstructing the other vessel. Blood flow into the jailed vessel would be compromised as would future access into and treatment in the distal portion of the jailed vessel.

Alternatively, access into a jailed vessel can be attained by carefully placing a guide wire through the stent and subsequently tracking a balloon catheter through the stent struts. The balloon could then be expanded, thereby deforming the stent struts and forming an opening into the previously jailed vessel. The cell to be spread apart is currently randomly and blindly selected by crossing a deployed stent with a guide wire. The drawback with this approach is that there is no way to determine or guarantee that the main-vessel stent struts are properly oriented with respect to the side branch or that an appropriate stent cell has been selected by the wire for dilatation. The aperture created often does not provide a clear opening and creates a major distortion in the surrounding stent struts. This may prevent the option of placing a stent in the jailed vessel. This technique also causes stent deformation to occur in the area adjacent to the carina, pulling the stent away from the vessel wall and partially obstructing flow in the originally non-jailed vessel. Deforming the stent struts to regain access into the previously jailed vessel is also a complicated and time consuming procedure associated with attendant risks to the patient. It is typically performed only if considered an absolute necessity. Vessels which supply a considerable amount of blood to the myocardium and may be responsible for the onset of angina or a myocardial infarct typify what would necessitate the subsequent strut deformation in order to reestablish blood flow into the vessel. The risks of procedural complications during this subsequent deformation are considerably higher than stenting in normal vessels. The inability to place a guide wire through the jailed lumen in a timely fashion could restrict blood supply and begin to precipitate symptoms of angina or even cardiac arrest. In addition, disturbed hemodynamics and subsequent thrombus formation at the jailed site could further compromise blood flow into the side branch.

Plaque shift is also a phenomena which is of concern when deploying a stent across a bifurcation. Plaque shift occurs when treatment of disease or plaque in one vessel causes the plaque to shift into another location. This is of greatest concern when the plaque is located on the carina or the apex of the bifurcation. During treatment of the disease the plaque may shift from one side of the carina to the other thereby shifting the obstruction from one vessel to the other vessel.

In another prior art method of implanting conventional stents in bifurcations, a “T” stent procedure includes implanting a stent in the side branch ostium of the bifurcation followed by stenting the main vessel across the side branch and subsequently deforming the struts as previously described, to allow blood flow and access into the side branch vessel. Alternatively, a stent is deployed in the parent vessel and across the side branch origin followed by subsequent strut deformation as to access the side branch as previously described, and finally a stent is placed into the side branch vessel. T stenting may be necessary in some situations in order to provide further treatment and additional stenting in the side branch vessel. This is typically necessitated when the disease is concentrated at the origin of the jailed vessel. This procedure is also associated with the same issues and risks previously described when stenting only one vessel and deforming the struts through the jailed vessel.

In another prior art method for treating bifurcated vessels, commonly referred to as the “Culotte technique,” the side branch vessel is first stented so that the stent protrudes into the main or parent vessel. A dilatation is then performed in the main or parent vessel to open and stretch the stent struts extending across the lumen from the side branch vessel. Thereafter, a stent is implanted in the main branch so that its proximal end overlaps with the portion of the first stent in the parent vessel. One of the drawbacks of this approach is that the orientation of the stent elements protruding from the side branch vessel into the main vessel is completely random. In addition excessive metal coverage exists from overlapping strut elements in the parent vessel proximal to the carina area. Furthermore, the deployed stent must be recrossed with a wire blindly and arbitrarily selecting a stent cell. When dilating the main vessel the stent struts are randomly stretched, thereby leaving the possibility of restricted access, incomplete lumen dilatation, and major stent distortion.

In another prior art procedure, known as “kissing” stents, a stent is implanted in the main vessel with a side branch stent partially extending into the main vessel creating a double-barrelled lumen of the two stents in the main vessel proximal to the bifurcation. Another prior art approach includes a so-called “trouser legs and seat” approach, which includes implanting three stents, one stent in the side branch vessel, a second stent in the distal portion of the main vessel, and a third stent, or a proximal stent, in the main vessel just proximal to the bifurcation.

More recently, bifurcated stents have been developed for delivery and deployment by bifurcated balloon catheters that allow for the simultaneous stenting of an entire bifurcation with no jailing, gaps nor overlaps. U.S. Pat. No. 6,802,856 is hereby incorporated by reference in its entirety. Such an approach however adds an additional degree of complexity as the stent must be dimensioned so as to fit the angle of the particular bifurcation being treated. Additionally, the flexibility of such stents may be somewhat compromised making it more difficult to advance the mounted stent through convoluted vasculature and to precisely position the device at the treatment site.

A stent system including stent and balloon configurations is needed that makes it easier to advance the assembly through the vasculature, to properly position the device at the diseased bifurcation, to accommodate a wide range of bifurcation angles and to simplify its deployment than stent systems that have previously been used to treat bifurcations.

### SUMMARY OF THE INVENTION

The invention provides for an improved stent system that facilitates the treatment of a bifurcation. The system includes multiple stents mounted on multiple balloons that provide for a highly flexible assembly that is easily advanceable into position. Additionally, the system readily conforms to any bifurcation angle and can further be readily tailored to accommodate any special requirements of a particular bifurcation site.

The stent system of the present invention employs two balloon catheters that are arranged in parallel. A first stent is mounted about both balloons, while a second stent is mounted about just one of the balloons and a third stent is mounted about the other of the two balloons. The second and third stents are positioned adjacent to one another and distal to the first stent. One or both of the distal balloons may optionally be deleted. The stents are preferably structurally independent of one another so as to maximize flexibility although a single link or weld may be relied upon to couple the proximal stent with one or both of the distal stents in an effort to maintain relative stent position during deployment.

The stent system of the present invention is not only able to conform to most any bifurcation angle but allows stents of different lengths, expanded diameters, strengths, patterns, materials and coatings to be combined so as to accommodate any special requirements of a particular treatment site.

These and other advantages of the present invention will become apparent from the following detailed description of preferred embodiments which, taken in conjunction with drawings, illustrate by way of example the principles of the invention.

### BRIEF DESCRIPTION OF THE DRAWINGS

FIG. 1 is a semi-schematic illustration of a preferred embodiment of the present invention;

FIG. 2 is a semi-schematic illustration of an alternative preferred embodiment of the present invention;

FIG. 3 is a semi-schematic illustration of another alternative preferred embodiment of the present invention;

FIG. 4 is a semi-schematic illustration of an additional alternative preferred embodiment of the present invention; and

FIGS. 5-7, in semi-schematic fashion, show the sequence of steps in the deployment of the system of the present invention at a bifurcation.

### DETAILED DESCRIPTION OF THE PREFERRED EMBODIMENTS

The present invention comprises a stent system that includes multiple stents mounted on multiple balloon catheters for use in treating a bifurcation. The stent system readily conforms to any bifurcation angle and the characteristics of the individual stents can readily be tailored to the satisfy the requirements of a particular treatment site.

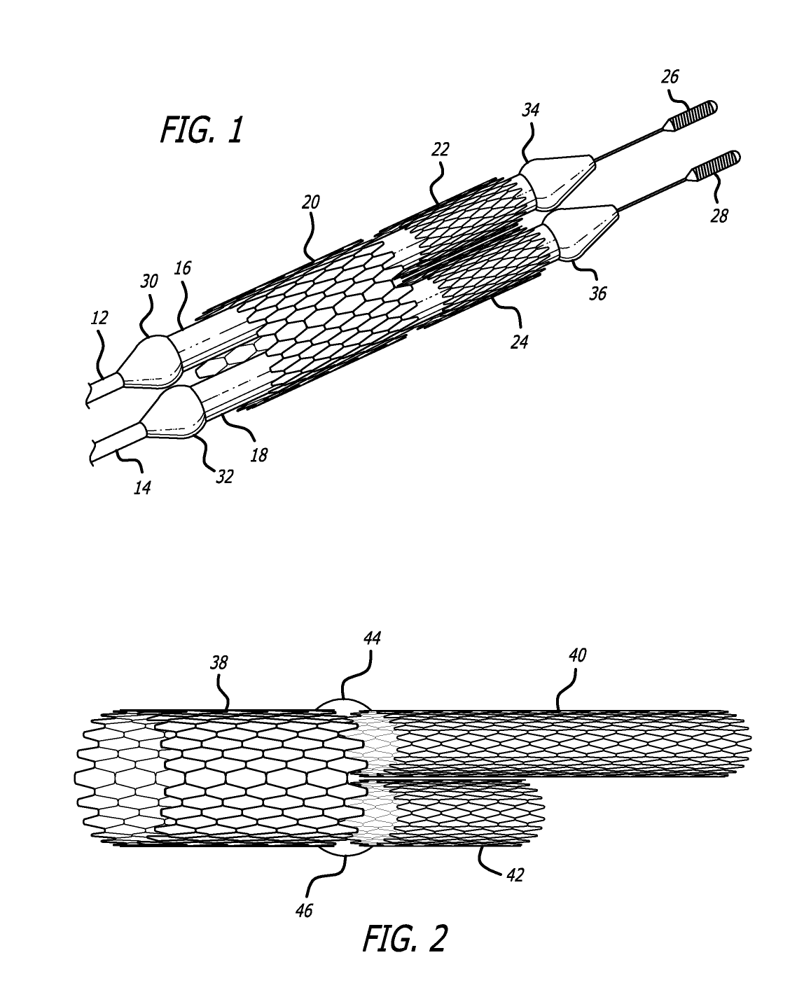

FIG. 1 is a semi-schematic illustration of a preferred embodiment of the present invention. Two balloon catheters 12, 14 are arranged so as to extend parallel one another and such that the respective inflatable balloons 16, 18 are positioned adjacent to one another. A first stent 20 is positioned about both balloons while a second stent 22 and a third stent 24 are respectively positioned about one of the individual balloon catheters. The second and third stents are positioned adjacent to one another and distal to the first stent. Each stent is structurally independent of the other stents and are not linked in any way.

The balloon catheters are configured in a conventional manner to the extent they include an internal guide wire lumen for receiving a guide wire 26, 28. The system can rely on any of a number of guide wire configurations, including Rapid Exchange, Over-The-Wire or even a combination of both. Additionally, the balloon catheters each include an inflation lumen through which the balloon is inflated and deflated. The inflation lumens can be plumbed such that the balloons are simultaneously or separately inflatable. The balloons may additionally include extra-high profile radiopaque markers which cause bulges 30, 32, 34, 36 to form in the deflated balloons which in turn serve as bumpers to help secure the stents to the balloons and prevent their shifting during advancement through the vasculature. Additional radiopaque markers can optionally be placed between stents so as to serve as corina markers.

Any number of stent configurations may be employed in the stent system of the present invention. Particular ring and link patterns, element dimensions and materials of manufacture can be selected to accommodate a particular application including, but not limited to, variations relating to length, expanded diameter, coverage, flexibility and even resorbability. The stent or stents may also be coated with any of the many different compositions that are well known in the art. All three stents may be similar or each stent may be different depending upon the application.

In adapting the embodiment illustrated in FIG. 1 to the treatment of the Left Main Bifurcation, the stents are each about 5 mm in length for an overall length of 10 mm. Such configuration is intended to cover a minimal amount of vessel length proximal and distal to the bifurcation. Upon inflation of the balloons and expansion of all three stents, secondary stents can be placed as needed. The extra high profile balloon markers which may be located outside the margins of the stents, under the extended length balloon tapers serve as “bumpers” to prevent the stents from shifting relative to one another and relative to the balloons. The short length and extremely proximal target anatomy could tend to enhance self-alignment of the stent into the bifurcation. The stent patterns may preferably be selected such that the proximal stent has greater radial strength than the two distal stents for this particular application as the left main segment may need more support than the branch segments.

FIG. 2 illustrates an alternative preferred embodiment of the present invention. The stents are shown sans catheters for clarity. This particular embodiment shows a differentiation in terms of the lengths of the individual stents. Not only is the proximal stent 38 shorter than the distal stents 40, 42, but the distal stents are of different length relative to one another as well. Additionally, this particular embodiment includes a single link 44, 46 that extends between the proximal stent and each distal stent. Such link may comprise a longitudinally extending element, suture, or merely a weld between adjacent stent components. Such links serve to maintain a relative position between the stents while the system spreads to conform to a bifurcation and during expansion.

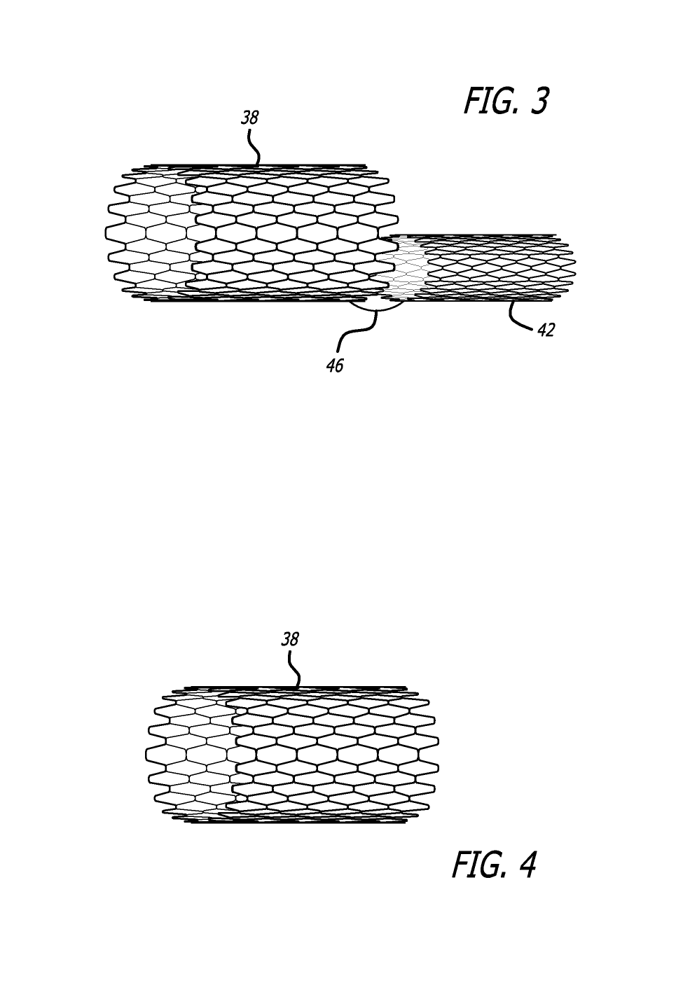

FIGS. 3 and 4 illustrate further alternative preferred embodiments of the present invention. The stents are again shown sans catheters for clarity. FIG. 3 illustrates an embodiment with only a single distal stent while the embodiment shown in FIG. 4 is devoid of any distal stents.

The balloons and stents of the stent system of the present invention is manufactured in the conventional manner. The assembly of the system requires the stents to be threaded onto the balloons and then crimped in place to substantially reduce the cross-section of the device.

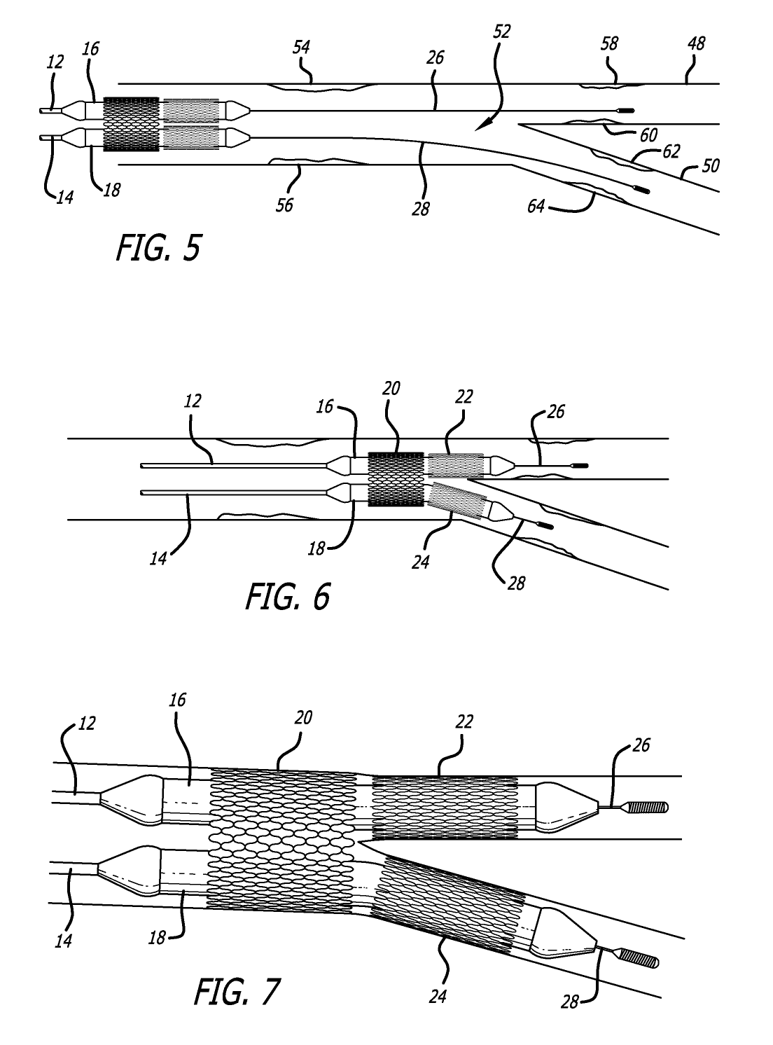

As is illustrated in FIG. 5, the system is deployed by first advancing the guide wires 26, 28 into place within the branches 48, 50 of the bifurcation 52. Plaque 54, 56, 58, 60, 62, 64 is shown as being present in multiple areas of the bifurcation. Once the guide wires are in place, the stent carrying balloon catheters 12, 14 are advanced into position as is shown in FIG. 6. Inflation of the balloons 16, 18 causes the stents to expand and compresses the plaque against the vessel walls as is shown in FIG. 7. Subsequent deflation of the balloons and retraction of the catheters leaves the stents in place to maintain blood flow therethrough.

While particular forms of the invention have been illustrated and described, it will be apparent to those skilled in the art that various modifications can be made without departing from the scope of the invention. Accordingly, it is not intended that the invention be limited except by the appended claims.

We claim:

1. A stent system for treating a bifurcated vessel having a main vessel and two branch vessels, comprising:
a first catheter having an expandable balloon;
a second catheter having an expandable balloon arranged such that said balloon of said first catheter is adjacent said balloon of said second catheter;
a first expandable stent disposed about both said balloon of said first catheter and said balloon of said second catheter;
a second expandable stent, disposed about the balloon of one of said catheters and distal to said first expandable stent.

2. The stent system of claim 1, further comprising a third expandable stent disposed about the balloon of the other of said catheter, distal to said first expandable stent and adjacent to said second expandable stent.

3. The stent system of claim 1, wherein said first catheter and said second catheter are joined to one another at a location proximal to said balloons.

4. The stent system of claim 1, wherein said first expandable stent is expandable to a larger diameter than said second expandable stent.

5. The stent system of claim 1, wherein said first expandable stent is longer than said second expandable stent.

6. The stent system of claim 1, wherein said first expandable stent is shorter than said second expandable stent.

7. The stent system of claim 2, wherein said first expandable stent is expandable to a larger diameter than said second and third expandable stents.

8. The stent system of claim 2, wherein said first expandable stent is longer than said second and third expandable stents.

9. The stent system of claim 1, wherein said first and second expandable balloons are of equal diameter.

10. The stent system of claim 1, wherein said first and second expandable balloons are of dissimilar diameter.

11. The stent system of claim 1, wherein said first and second expandable balloons are of similar length.

12. The stent system of claim 1, wherein said first and second expandable balloons are of dissimilar lengths.

13. The stent system of claim 1, wherein said first and second expandable balloons are of similar diameter and length.

14. The stent system of claim 1, wherein said first and second expandable balloons are of dissimilar diameter and length.

15. The stent system of claim 1, further comprising a single link extending between said first stent and said second stent.

16. The stent system of claim 2, further comprising a first single link extending between said first stent and said second stent and a second single link extending between said first stent and said third stent.

17. The stent system of claim 16, wherein said first and second links are arranged so as to be diametrically opposed to one another.

18. A stent system for treating a bifurcated vessel having a main vessel and two branch vessels, comprising:
a first catheter having an expandable balloon;
a second catheter having an expandable balloon arranged such that said balloon of said first catheter is adjacent said balloon of said second catheter; and
a first expandable stent disposed about both said balloon of said first catheter and said balloon of said second catheter.

19. A method of stenting a bifurcation within a vasculature, comprising:
providing a pair of expandable balloons mounted on catheters;
mounting a first stent so as to encompass both said balloons;
mounting a second stent distal to said first stent and so as to encompass one of said balloons;
advancing said balloon mounted stents through said vasculature to said bifurcation such that one of said pair of balloons enters one branch of said bifurcation and the other of said pair of balloons with said second stent mounted thereon enters the other branch of said bifurcation;
inflating said balloons so as to expand said stents;
deflating said balloons; and
retracting said catheters.

20. A method of stenting a bifurcation within a vasculature, comprising:
providing a pair of expandable balloons mounted on catheters;
mounting a first stent so as to encompass both said balloons;
mounting a second stent distal to said first stent and so as to encompass one of said balloons;
mounting a third stent distal to said first stent, adjacent to said second stent so as to encompass the other of said balloons;
advancing said balloon mounted stents through said vasculature to said bifurcation such that one of said pair of balloons enters one branch of said bifurcation and the other of said pair of balloons with said second stent mounted thereon enters the other branch of said bifurcation;
inflating said balloons so as to expand said stents;
deflating said balloons; and
retracting said catheters.

21. The method of claim 20, wherein said balloons are inflated simultaneously.

22. The method of claim 20, wherein said balloons are inflated separately.

23. The method of claim 22, wherein said balloons are inflated to similar diameters.

24. The method of claim 22, wherein said balloons are inflated to different diameters.
